(19) United States
(12) Patent Application Publication
(10) Pub. No.: US 2019/0316083 A1
(43) Pub. Date: Oct. 17, 2019

(54) CULTURE METHOD FOR LONG-TERM MAINTENANCE AND PROLIFERATION SUBCULTURE OF HUMAN HEPATOCYTES

(71) Applicant: THE SECOND MILITARY MEDICAL UNIVERSITY AFFILIATED EASTERN HEPATOBILARY SURGERY HOSPITAL, Shanghai (CN)

(72) Inventors: Peilin ZHANG, Shanghai (CN); Hongyang WANG, Shanghai (CN)

(21) Appl. No.: 16/455,578

(22) Filed: Jun. 27, 2019

Related U.S. Application Data
(62) Division of application No. 15/314,923, filed on Nov. 29, 2016.

(30) Foreign Application Priority Data
May 30, 2014 (CN) 201410239238.X

(51) Int. Cl. C12N 5/071; C12N 5/00

(52) U.S. Cl. CPC C12N 5/067; C12N 5/0018; C12N 2501/135; C12N 2501/237; C12N 2501/115; C12N 2501/727; C12N 2501/39; C12N 2501/12; C12N 2501/235; C12N 2501/11; C12N 2501/15

(57) ABSTRACT

A method for culturing hepatocytes involves maintenance, proliferation, and passaging of the hepatocytes. The method includes culturing the hepatocytes in a culture medium, which includes a hepatocyte basic medium; CHIR99021, which is a GSK-3 beta inhibitor at a concentration of 0.5-10 μM, SB 431542 or A83-01, which are RGF beta inhibitors, at a concentration of 0.5-10 μM, N-acetyl-cysteine at a concentration of 0.25-25 mM; and Oncostatin M at a concentration of 1-100 ng/mL There is no exogenous gene introduced into the hepatocytes, and the genetic background of the hepatocytes is not changed.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

The present invention relates to the field of biology and medicine; more particularly, the present invention relates to specific culture medium and culturing method for human hepatocytes in vitro long-term maintenance and proliferation/passaging.

### Description of the Related Art

According to the world health organization statistics, worldwide every year, millions of people die from liver diseases. China is a country with big incidence of liver disease, wherein only hepatitis b and c virus carriers reach 0.14 billion, accounting for about 28% of the whole world, and there are 297 thousands of liver cancer patients, accounting for more than half of the world (55%). Therefore, in aspects such as liver disease research, new medicine liver toxicity detection, biological artificial liver and hepatocyte transplantation and the like, there is a need in a large number of qualified human hepatocytes. However, lacking of liver supply is a global problem, while the existing human hepatocyte culturing methods all have deficiencies that conventional culture can only maintain a short period of its morphology and function, and the unconventional culture operations are very complicated, which have high requirements and have various interference factors, with short culture time and neither can achieve proliferation and passage. Therefore, it is desirable to develop new human hepatocyte culture proliferation and passage method.

Human hepatocytes are widely used in medicine and related fields. Domestic and foreign scholars have done large amount of research on hepatocyte culture method for a long time. However, there is still no report on the methods for long-term culture of hepatocytes and retaining their morphology and function. Particularly, there is no breakthrough in the proliferation and passage under hepatocyte in vitro culture conditions. At present, isolated human hepatocytes can only last up to 14 days in in vitro conventional culture, and other methods such as symbiotic culture, suspension culture, sandwich culture, three-dimensional culture and the like can culture hepatocytes up to 3 months. However, due to various deficiencies such as interference factors (such as in symbiotic culture), complex operation (sandwich culture), or unable to directly observe the cell morphology changes (suspension culture and three-dimensional culture), they cannot satisfy the need of liver disease research and can only be used partially for detecting liver toxicity of new drugs. However, even if they are used in detection of new drug liver toxicity, due to the presence of the above defects, the results are only relevantly meaningful and valuable. More seriously, the existing human hepatocyte culture methods are not able to proliferate and passage. Accordingly, not only that the conventional method and the culture hepatocytes cannot be used as qualified cell model for the liver disease studies, they are also less likely to provide qualified hepatocyte supply for constructing biological artificial liver and hepatocyte transplantation.

In view of the above, it is desirable to develop novel culture method and culture medium for long-term human hepatocyte maintenance, and proliferation and passage.

### SUMMARY OF THE INVENTION

An object of the present invention is to provide specific culture mediums and culturing methods for human hepatocytes long-term maintenance, proliferation and passaging.

In a first aspect of the present invention, it is provided a culture medium for culturing human hepatocyte, which comprises: hepatocyte basic medium; and

GSK-3 beta inhibitor CHIR99021: 0.5-10 μM;

TGF beta inhibitor SB 431542: 0.5-10 μM; and/or

TGF beta inhibitor A83-01: 0.5-10 μM.

In a preferred embodiment, the culture medium comprises:

GSK-3 beta inhibitor CHIR99021: 1-4 μM;

TGF beta inhibitor SB 431542: 1-8 μM; and/or

TGF beta inhibitor A83-01: 1-8 μM.

In another preferred embodiment, the culture medium further comprises ingredients selected from the group consisting of:

N-acetyl-cysteine (NAC): 0.25-25 mM;

Oncostatin M (OSM): 1-100 ng/mL; and/or

Leukemia inhibitory factor (LIF): 100-1000 u/mL.

In another preferred embodiment, the culture medium comprises:

N-acetyl-cysteine (NAC): 0.5-12.5 mM;

Oncostatin M (OSM): 5-80 ng/mL; and/or Leukemia inhibitory factor (LIF): 150-800 u/mL.

In another preferred embodiment, the culture medium further comprises ingredients selected from the group consisting of

| | | |
|---|---|---|
| human epidermal growth factor | 5-100 | ng/ml; |
| Human fibroblast growth factor | 5-100 | ng/ml; |
| human hepatocyte growth factor | 5-100 | ng/ml; |
| dexamethasone | 0.05-1 | μM; |
| platelet derived factor | 1-100 | ng/ml; |
| Triiodothyronine | 1-100 | ng/ml. |

In another preferred embodiment, the culture medium comprises:

| | | |
|---|---|---|
| human epidermal growth factor | 5-50 | ng/ml; |
| Human fibroblast growth factor | 5-50 | ng/ml; |
| human hepatocyte growth factor | 5-50 | ng/ml; |
| dexamethasone | 0.1-0.8 | μM; |
| platelet derived factor | 5-50 | ng/ml; |
| Triiodothyronine | 20-80 | ng/ml. |

In another embodiment, the hepatocyte basic medium is a basic cell culture medium supplemented with 2.5% N2, 5% B27, 1% non-essential amino acids, 1% sodium pyruvate, and penicillin-streptomycin mixed solution (100×); or with: 5% N2 or 10% B27, 1% non-essential amino acids, 1% sodium pyruvate, and 1% penicillin-streptomycin (100×), etc. Or the hepatocyte basic medium is directly replaced by basal cell culture medium. The basal cell culture medium can be such as DMEM/F12, MEM, DMEM, RPMI 1640, Neuronal basal or Fischers, etc., which are all commercially available products.

In another aspect of the present invention, it is provided a kit for culturing human hepatocytes; the kit comprises any one of the above-mentioned culture medium.

In another aspect of the present invention, it is provided the use of the culture medium or the kit, for maintenance, proliferation, and passaging of human hepatocytes.

In another aspect of the present invention, it is provided a method for maintenance, proliferation, and passaging of hepatocytes, the method comprising the steps of: culturing hepatocytes using any one of the above culture medium. Preferably, the method for culturing hepatocytes comprises the steps of:

(1) Backing the culture plate (preferably, with one of matrigel, rat-tail collagen, gelatin, fibronectin, vitronectin) for 1-24 hours, mixing and suspending the primary hepatocytes in adherent culture medium (preferably, as hepatocyte basic medium supplemented with 10% serum); plating, and incubating at 37° C. for 1-12 hours;

(2) Discarding culture medium in (1), and transferring the hepatocytes into any one of the culture medium mentioned in claims 1-5.

In another preferred embodiment, in the previously described culture medium, kit, use or method, the human hepatocytes include, but are not limited to: the human primary hepatocytes and the hepatocytes passaged therefrom, human hepatocytes obtained from stem cells or human hepatocytes transformed from adult cells.

Method and other aspects of the present invention, in light of the disclosure herein, will be apparent to those skilled in this art.

### BRIEF DESCRIPTION OF THE DRAWINGS

FIG. 1 shows the human primary hepatocytes cultured under different conditions, cell morphological photo taken on 1, 14 and 20 days.

FIG. 2 shows cell morphological photo, in vitro cultured with Hepatocyte culture medium I-2.1 for 14 days.

FIG. 3 shows cell morphological photo, in vitro cultured with Hepatocyte culture medium I-3.4 for 21 days.

FIG. 4 shows cell morphological photo, in vitro cultured with Hepatocyte culture medium I-3.1 for 35 days.

FIG. 5 shows cell morphological photo, 2^{nd} generation of passage, cultured with Hepatocyte culture medium I-3.2 for 56 days in total.

FIG. 6 shows cell morphological photo, 2^{nd} generation of passage, cultured with Hepatocyte culture medium I-3.3 for 80 days in total.

FIG. 7 shows cell morphological photo, 3^{rd} generation of passage, cultured with Hepatocyte culture medium I-2.2 for 110 days in total.

FIG. 8 shows cell morphological photo of hepatocytes passaged in Hepatocyte culture medium I-2.2.

FIG. 9 shows cell morphological photo of human primary hepatocytes, taken on day 20, cultured in Hepatocyte culture medium I-1.1/2 and 1-2.1/2, wherein “2 core factors” means “CHIR99021+SB431542” or “CHIR99021+A83-01”.

FIG. 10 shows immunostaining of liver-specific marker, cultured in hepatocyte culture medium I-2.2 for 84 days.

FIG. 11 shows expression of liver related genes, cultured in hepatocyte culture medium I-2.1 for 77 days.

FIG. 12 shows assay result of p450 enzyme induction function, culture for 11 weeks in hepatocyte culture medium I-3.1

FIG. 13 shows hepatocyte glycogen staining result, cultured for 11 weeks in Hepatocyte culture medium I-3.1.

### DETAILED DESCRIPTION OF THE PREFERRED EMBODIMENT

The present inventors, through long-term and intensive study, disclosed herein a culture medium and method for long-term maintenance, proliferation, and passaging of human hepatocytes. The method overcomes the defects of the existing hepatocyte culture method. The method can maintain cultured human hepatocytes for a long time under conventional conditions, and make them proliferate and passage, while retaining substantially consistent morphology and function as freshly isolated human primary hepatocytes. The method uses simple culture conditions, and is low cost, safe and stable.

As used herein, the term “comprises” or “comprising”, includes “including”, “mainly composed of (made of)”, “essentially consisting of” and “consisting of”.

Culture Medium

The inventor provides human hepatocyte culture medium for culturing human hepatocytes. The culture medium comprises GSK3 beta inhibitor CHIR99021, TGF beta inhibitor SB 431542 or/and A83-01. The above-mentioned components are added into the hepatocyte basic medium with suitable ratios, to provide suitable growth environment to hepatocytes, promote hepatocyte growth and expansion, and maintain the hepatocyte morphology and function in a relatively long period of time (about 15 days). In a preferable embodiment of the invention, the amounts of the components of the present culture medium are shown in table 1.

TABLE 1

| | | Amount | Preferred amount |
|---|---|---|---|
| GSK3 beta inhibitor CHIR99021 | | 0.5-10 μM | 1-4 μM |
| At least one TGF beta | SB431542 | 0.5-10 μM | 1-8 μM |
| inhibitor | A83-01 | 0.5-10 μM | 1-8 μM |

The GSK3 beta inhibitor CHIR99021 has the structural formula of the following formula (I):

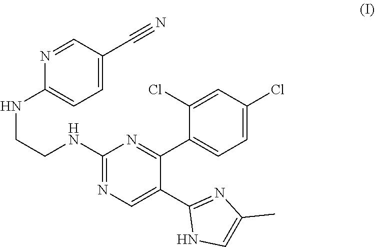 [SMILES: Cc1c[nH]c(-c2cnc(NCCNc3ccc(C#N)cn3)nc2-c2ccc(Cl)cc2Cl)n1]

The TGF beta inhibitor SB 431542 has the structural formula of the following formula (II):

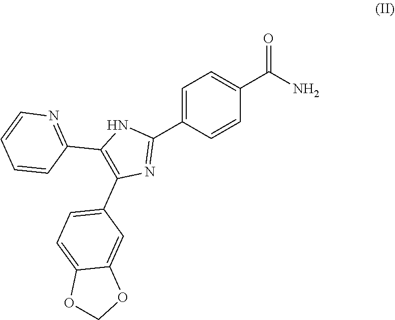 [SMILES: NC(=O)c1ccc(-c2nc(-c3ccc4c(c3)OCO4)c(-c3ccccn3)[nH]2)cc1]

The TGF beta inhibitor A83-01 has the structural formula of the following formula (III):

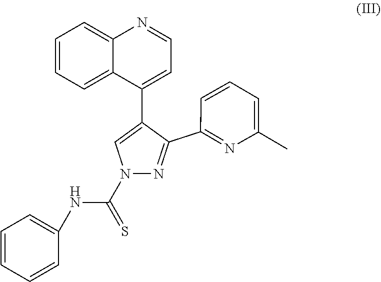 [SMILES: Cc1cccc(-c2nn(C(=S)Nc3ccccc3)cc2-c2ccnc3ccccc23)n1]

The present invention also includes the equivalent of medical product, analogs, and/or the salt, hydrate or precursor of the above-described compounds (I), (II) or (III).

“Analogs” of the compound include, but are not limited to: isomers and racemates of the compound. The compounds have one or more asymmetric centers. Therefore, these compounds may be presented as racemic mixtures, individual enantiomers, individual diastereomers, diastereomeric mixtures and cis- or trans-isomers.

The “salts” include, but are not limited to: (1) salts formed with an inorganic acid such as hydrochloric acid, sulfuric acid, nitric acid, phosphoric acid and the like; (2) salts formed with organic acids, such as acetic acid, oxalic acid, succinic acid, tartaric acid, methanesulfonic acid, maleic acid, or arginine and the like. Other salts include salts formed with alkali metals or alkaline earth metals, such as sodium, potassium, calcium or magnesium.

The “compound precursor” refers to compound precursors that can be transformed into one compound of compound (I), (II) or (III), or the salt or solution thereof in the culture medium after applied or processed by suitable method.

In a preferable embodiment of the invention, the culture medium further comprises one or more components selected from table 2: N-acetyl-cysteine (NAC), oncostatin m (OSM), leukemia inhibitory factor (LIF). The components are added at suitable ratios to hepatocyte culture medium comprising GSK3 beta inhibitor CHIR99021, TGF beta inhibitor SB 431542 and/or A83-01, providing good culture conditions for the proliferation of hepatocytes, further extending the maintenance of hepatocyte culture, and achieving long-term maintenance, proliferation, and passaging. The hepatocytes obtained through the maintenance, proliferation, and passaging can stably last for more than 4 months and keeping substantially consistent morphology and function with the freshly isolated hepatocytes.

TABLE 2

| | Amount | | Preferred Amount | |
|---|---|---|---|---|
| N-acetyl-cysteine (NAC) | 0.25-25 | mM | 0.5-12.5 | mM |
| Oncostatin M(OSM) | 1-100 | ng/ml | 5-80 | ng/ml |
| leukemia inhibitory factor(LIF) | 100-1000 | u/ml | 150-800 | u/ml |

In a preferable embodiment of the invention, the culture medium further comprises one or more components selected from table 3: human epidermal growth factor, human fibroblast growth factor, human hepatocyte growth factor, dexamethasone, platelet-derived factor, triiodothyronine. These components are added at suitable ratios to hepatocyte culture medium comprising GSK3 beta inhibitor CHIR99021, TGF beta inhibitor SB 431542 or/and A83-01 (preferably further comprising components selected from the group consisting of n-acetyl-cysteine, oncostatin M, and leukemia inhibitory factor), so as to further enhance the growth and proliferation of hepatocytes and improve the biological behavior and functionality under the effect of these factors. As factors known in the art, the functions of the factors listed in table 3 are already well known to those skilled in the art. Therefore, after processing with these factors, the biological behavior and functionality of the hepatocytes are also known in the art.

TABLE 3

| | Amount | | Preferred Amount | |
|---|---|---|---|---|
| human epidermal growth factor | 5-100 | ng/ml | 5-50 | ng/ml |
| Human fibroblast growth factor | 5-100 | ng/ml | 5-50 | ng/ml |
| human hepatocyte growth factor | 5-100 | ng/ml | 5-50 | ng/ml |
| dexamethasone | 0.05-1 | μM | 0.1-0.8 | μM |
| platelet derived factor | 1-100 | ng/ml | 5-50 | ng/ml |
| Triiodothyronine | 1-100 | ng/ml | 20-80 | ng/ml |

The components in Table 1, preferably further with the components in the formulations of Table 2 and/or Table 3 are dissolved in hepatocyte basic medium according to the final concentration mentioned in the tables, to provide a suitable environment for long-term maintenance, proliferation and passaging of hepatocytes.

The hepatocyte basic medium is a basal cell culture medium supplemented with: 2.5% N2, 5% B27, 1% non-essential amino acids, 1% sodium pyruvate, and 1% penicillin-streptomycin mixed solution (100×); or supplemented with: 5% N2 or 10% B27, 1% non-essential amino acids, 1% sodium pyruvate, and 1% penicillin-streptomycin (100×), etc.

The basal cell culture medium is DMEM/F12, MEM, DMEM, RPMI 1640, Neuronal basal or Fischers, etc., which are commercially available products. It should be understood that the basal cell culture medium is known culture medium readily available to those skilled in the art.

The culture medium optimized by the inventors comprises sufficient and reasonable components for maintaining hepatocyte growth or promoting hepatocyte expansion, thereby being beneficial to hepatocyte culture.

The components for formulating the culture medium are readily available to those skilled in the art. For example, they can be purchased commercially, or they can be obtained by artificial synthesis or recombinant expression.

Culture Method

The present invention also provides a method of culturing hepatocytes, the method comprising: culturing hepatocytes using any one of the above culture medium.

In the present invention, the “human hepatocyte” includes human hepatocytes from any source, including but not limited to, the human primary hepatocytes and the hepatocytes passaged therefrom, human hepatocytes derived from stem cells or human hepatocytes transformed from adult cells. For example, if desired, a two-step perfusion method can be used to separate human primary hepatocytes from surgically resected liver tissue.

The skilled in the art can refer to the existing hepatocyte isolation techniques to obtain the desired hepatocyte using the preferred mediums provided by the present invention.

In a preferable embodiment of the invention, a method for maintaining and culturing hepatocytes comprises the steps of: (1) Backing the culture plate with one of the material selected form matrigel, rat-tail collagen, gelatin, fibronectin, vitronectin for 1 to 24 hours, mixing and suspending the primary hepatocytes in adherent culture (preferably, hepatocyte basic medium supplemented with 10% serum); plating, and incubating at 37° C. for 1 to 12 hours;

(2) Discarding culture medium in (1), and transferring the hepatocytes into any one of the culture medium mentioned in Claims 1-5.

Hepatocytes cultured by the present method can be passaged (including passaged repeatedly) at any time during culture period. As a preferred embodiment of the present invention, the subculture steps comprises the steps of digesting the cultured hepatocytes with digestion solution (selected from trypsin, EDTA, Acutase or trypleE etc.) to individual cell, resuspending, and then passaging at a ratio of 1:2 to 1:4, culturing the passaged hepatocytes according to the method of steps (1) and (2). Hepatocytes obtained by passaging can be further passaged at any time during culture period.

The hepatocytes cultured by the present methods can be used in any experiments for detecting the morphology and function. The maintained, proliferated, and passaged hepatocytes can stably last for more than 4 months and keep substantially consistent morphology and function with the freshly isolated hepatocytes.

Application

The human hepatocytes cultured by the method of the present invention not only retain the morphology and function of primary hepatocytes, but also with advantages such as easy and feasible operation, low cost and safe and stable application. The hepatocytes are optimal cell model for liver disease study, and also can be used in hepatotoxicity test for new drugs, bio-artificial liver and cell transplantation for treating liver diseases and the like, which have a wide range of applications, with significant research value, great social benefit and economic benefit.

In the existing conventional adherent culture method, the cultured hepatocytes will lose their morphology and function within a few hours to a few days, up to 14 days. In the existing non-conventional culture such as symbiotic culture, suspension culture, sandwich culture, three-dimensional culture, the cultured hepatocytes can be cultured and maintained for 2-3 months, and the longest time may be prolonged to 3 months. However, due to various interference factors (e.g., symbiotic-culturing), complicated operations (sandwich culture), or unable to directly observe the cell morphology changes (suspension culture and three-dimensional culture), these methods cannot satisfy the needs of liver disease research and only can be partially used for detecting the liver toxicity of new drugs. However, even if used for detecting liver toxicity of new drug, due to the presence of the various defects above, the results are also only having relative significance and value. Proliferation and passage of hepatocyte in vitro is not allowed in the existing hepatocyte culture methods mentioned above, while the present long-term culture, proliferation and passaging method for human hepatocyte overcomes all the defects of the aforementioned hepatocyte culture methods, with creative advantages such as common culturing, simple operation, easy observation, low cost, long culture and maintenance time (more than 4 months), and able to proliferation and passage, etc.

The hepatocytes cultured, propagated and passaged for a long-term by the present method not only can provide qualified hepatocyte model for liver disease research, in vivo regeneration, in vitro reconstruction of liver and hepatocyte de-differentiation and other studies provide sufficient qualified hepatocyte model; it can also provide human hepatocyte model and experimental platform for drug screening, drug target study, pharmacological analysis and liver toxicity evaluation, which is incomparable by animal model. More importantly, a sufficient number of hepatocytes may provide normal hepatocyte source for the hepatocyte transplantation and bio-artificial liver of liver disease patients, which may save a large number of patients.

The advantages of the present long-term culture, proliferation and passaging method mainly lie in:

1\grave{ } Operating conditions are conventional adherent culture conditions, simple to operate, easy to observe the morphological and functional changes of hepatocytes.

2\grave{ } No exogenous gene introduction, no change in genetic composition of hepatocytes, without interference from exogenous genes and without change in genetic background, genetically safe, with reliable experimental results.

3\grave{ } No other interfering factors, can faithfully reflect the experimental results.

4\grave{ } can maintain hepatocyte morphology and function in long-term (more than 4 months) culture, and can meet the requirements of fundamental experimental researches such as mechanism of liver disease, effective drug targets, drug toxicology, providing qualified hepatocyte model.

5\grave{ } can in vitro proliferate and passage, can provide hepatocytes of same batch for researches in need of more amount of hepatocytes to improve the comparability of the experimental control, can be qualified human hepatocyte resource for hepatocyte transplantation, bio-artificial liver, drug liver toxicity detection, etc.

The following examples further illustrate the invention. It should be understood that these examples are only intended to illustrate the invention, but not to limit the scope of the invention. In the following examples, the experiment methods without detailed conditions normally refer to those mentioned in J. Sambrook et al., eds., molecular cloning: a laboratory manual, third edition, academic press, 2002, or according to the conditions recommended by the manufacturer.

### Example 1, Preparation of Human Hepatocyte Culture Medium

1\grave{ } A hepatocyte basal medium was prepared according to conventional method, i.e., the components of table 4 were added into DMEM/F12 (basal cell culture medium) (in v/v %):

TABLE 4

| | |
|---|---|
| N2 | 2.5% |
| B27 | 5% |
| non-essential amino acids (Non-AA) | 1% |
| sodium pyruvate (Sodium pyruvate) | 1% |
| streptomycin mixed solution (100x) | 1% |

2\grave{ } In the above-described hepatocyte basic culture medium, respectively, components were added with final concentrations as shown in table 5, thereby obtaining hepatocyte culture medium I-1.1/2, I-2.1/2 and hepatocyte culture medium I-3.1/2/3/4.

TABLE 5

| | medium | medium | medium | | medium | | medium | | medium | | medium | | medium | |
|---|---|---|---|---|---|---|---|---|---|---|---|---|---|---|
| | I -1.1 | I -1.2 | I -2.1 | | I -2.2 | | I -3.1 | | I -3.2 | | I -3.3 | | I -3.4 | |
| GSK3βinhibitor | 2 μM | 2 μM | 1.5 | μM | 1.5 | μM | 3 | μM | 3 | μM | 3 | μM | 3 | μM |
| CHIR99021 | | | | | | | | | | | | | | |
| TGFβinhibitor | 2 μM | 0 | 0 | | 2 | μM | 5 | μM | 4 | μM | 0 | μM | 0 | μM |
| SB431542 | | | | | | | | | | | | | | |
| TGFβinhibitor A83-01 | 0 | 1 μM | 1.5 | μM | 0 | | 0 | | 0 | | 2.0 | μM | 1.5 | μM |
| N-acetyl-cysteine(NAC) | 0 | 0 | 1 | mM | 1.25 | mM | 3 | mM | 6 | mM | 1.25 | mM | 2.5 | mM |

| | | | | | | | | | | | | |
|---|---|---|---|---|---|---|---|---|---|---|---|---|
| Oncostatin M(OSM) | 0 | 0 | 5 | ng/ml | 10 | ng/ml | 0 | 20 | ng/ml | 40 | ng/ml | 0 |

| | | | | | | | | | | |
|---|---|---|---|---|---|---|---|---|---|---|
| leukemia inhibitory | 0 | 0 | 0 | 0 | 150 | u/ml | 0 | 0 | 800 | u/ml |
| factor (LIF) | | | | | | | | | | |

| | | | | | | | | | | |
|---|---|---|---|---|---|---|---|---|---|---|
| human epidermal | 0 | 0 | 0 | 0 | 0 | 5 | ng/ml | 10 | ng/ml | 0 |
| growth factor(EGF) | | | | | | | | | | |

| | | | | | | | | | | |
|---|---|---|---|---|---|---|---|---|---|---|
| Human fibroblast | 0 | 0 | 0 | 0 | 10 | ng/ml | 5 | ng/ml | 0 | 0 |
| growth factor(bFGF) | | | | | | | | | | |

| | | | | | | | | | |
|---|---|---|---|---|---|---|---|---|---|
| human hepatocyte | 0 | 0 | 0 | 0 | 0 | 0 | 10 | ng/ml | 0 |
| growth factor(HGF) | | | | | | | | | |

| | | | | | | | | | |
|---|---|---|---|---|---|---|---|---|---|
| platelet derived factor | 0 | 0 | 0 | 0 | 0 | 0 | 0 | 10 | ng/ml |

| | | | | | | | | | |
|---|---|---|---|---|---|---|---|---|---|
| Triiodothyronine | 0 | 0 | 0 | 0 | 0 | 0 | 40 | ng/ml | 0 |

| | | | | | | | | | |
|---|---|---|---|---|---|---|---|---|---|
| dexamethasone | 0 | 0 | 0 | 0 | 0 | 0 | 0 | 0.4 | μM |

### Example 2, Long-Term Proliferation, Passage and Maintenance of Human Hepatocyte Culture

1\grave{ } Culturing human primary hepatocytes with Hepatocyte culture medium I-1.1, hepatocyte culture medium I-1.2

(1) Primary hepatocytes (isolated product from ablated human liver tissue) were purchased from Life Technology, Inc.

(2) The culture plate was backed with matrigel (1:30) for 1 hour, then the primary hepatocytes were mixed and suspended in adherent culture medium (hepatocyte basal medium supplemented with 10% serum), prior to being plated and incubated at 37° C. for 1 hour;

(3) The culture medium of (2) was discarded, and the hepatocytes are swapped into hepatocyte culture medium I-1.1 and, I-1.2 for culture.

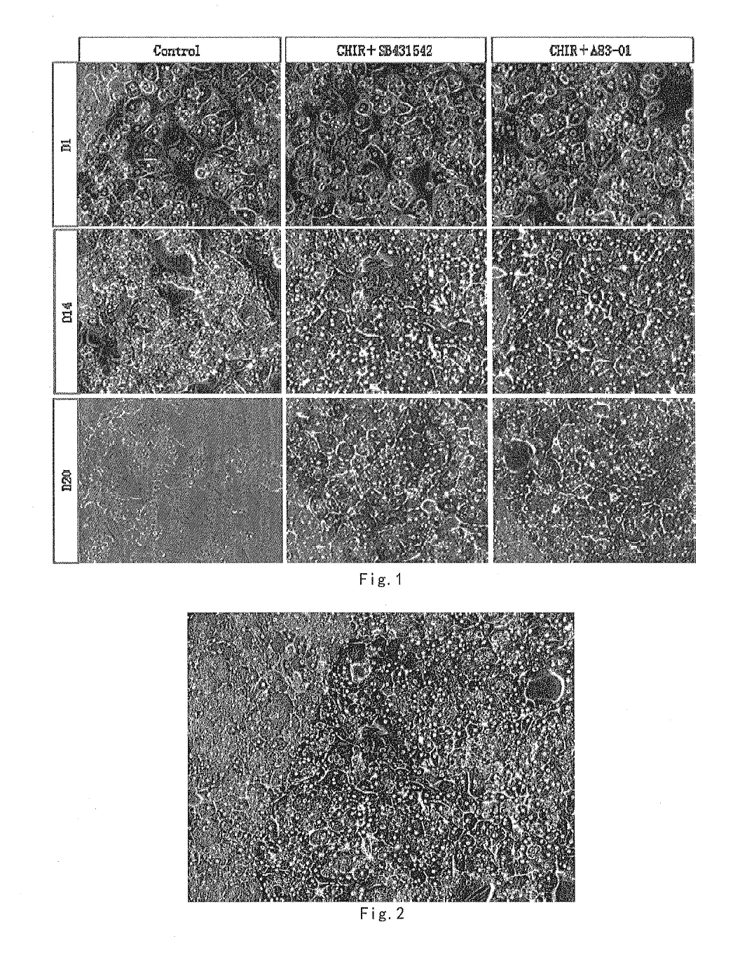

Cell morphology images were taken at 1, 14, 20 days of culture with hepatocyte culture mediums I-1.1 and I-1.2 (cell cultured without GSK3 beta inhibitor and TGF beta inhibitor was used as a control). The morphological image is as shown in FIG. 1.

As seen in FIG. 1, adding GSK3 beta inhibitor and TGF beta inhibitor into hepatocyte basic culture medium can keep the morphology of human hepatocytes close to that of freshly isolated hepatocyte in a relatively long period of time. The morphology was maintained at day 14 (D14) of the culture. However, the morphology of hepatocytes without addition of GSK3 beta inhibitors and TGF beta inhibitors could not be maintained for a long period of time.

2\grave{ } Culturing human primary hepatocytes with Hepatocyte culture medium I-2.1/2, hepatocyte culture medium I-3.1/2/3/4

(1) Primary hepatocytes were purchased from Life Technology, Inc.

(2) The culture plate was backed with matrigel (1:30) for 1 hour, then the primary hepatocytes were mixed and suspended in adherent culture medium (hepatocyte basal medium supplemented with 10% serum), prior to being plated and incubated at 37° C. for 1 hour;

(3) The culture medium of (2) was discarded, and the hepatocytes are swapped into hepatocyte culture medium I-2.1/2 and hepatocyte culture medium I-3.1/2/3/4 for culture.

The morphology image of the cells in vitro cultured for 14 days using hepatocyte culture medium I-2.1 was shown in FIG. 2.

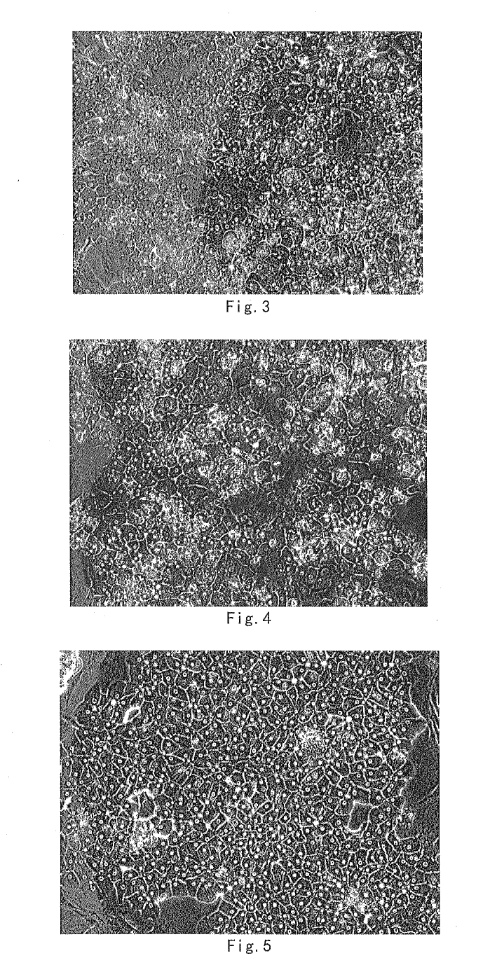

The morphology image of the cells in vitro cultured for 21 days using hepatocyte culture medium I-3.4 was shown in FIG. 3.

The morphology image of the cells in vitro cultured for 35 days using hepatocyte culture medium I-3.1 was shown in FIG. 4.

3. Passage culture of human primary hepatocytes

Passage Step:

Cultured hepatocytes were digested with digestion solution (pancreatin) into individual cells, resuspended and then passaged at a ratio of 1:2. The passaged cells were cultured according to steps (2) and (3) in “2”.

Hepatocytes were in vitro cultured with Hepatocyte culture medium I-3.2, then passaged to 2^{nd} generation at day 14, cultured for 56 days altogether. The cell morphological photo is shown in FIG. 5.

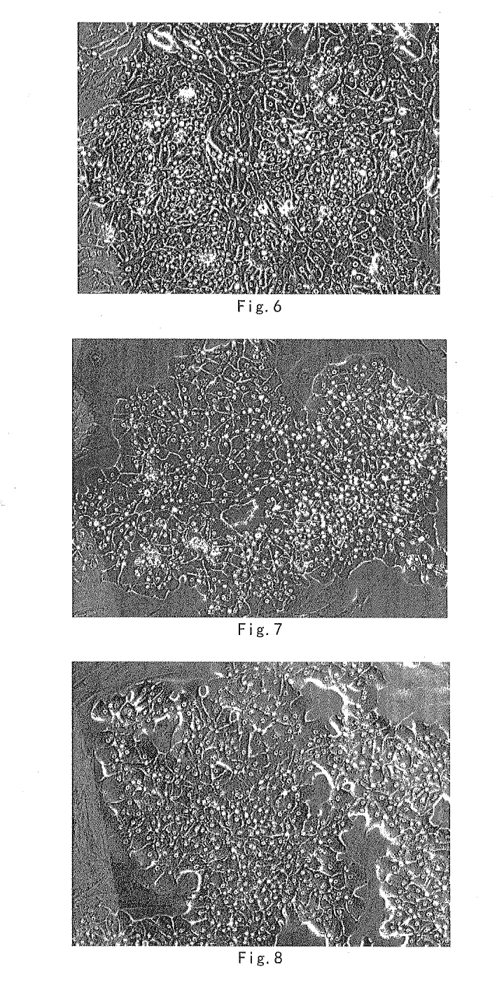

Hepatocytes were in vitro cultured with Hepatocyte culture medium I-3.3, then passaged to 2^{nd} generation at day 21 and passaged to 3^{rd} generation on day 35, cultured 80 days altogether. The cell morphological photo is shown in FIG. 6;

Hepatocytes were in vitro cultured with Hepatocyte culture medium I-2.2, passaged to 2^{nd} generation at day 21, passaged to 3^{rd} generation at day 35, and then passaged to 4^{th} generation at day 28, cultured for 110 days altogether. The cell morphological photo is shown in FIG. 7.

Hepatocytes cultured in Hepatocyte culture medium I-2.2 for 15 days, digested by pancreatin, passaged at ratio of 1:3, and then cultured according to step (2) and (3) in “2”. The cell morphological photo is shown in FIG. 8.

In view of the above, it can be seen that the hepatocytes could maintain the morphology and function close to the freshly isolated hepatocyte for a long time by adding GSK3 beta inhibitor and TGF beta inhibitors, and at the same time adding NAC, LIF or OSM in hepatocyte basic culture medium, and the proliferation and passage could be achieved. Further addition of one or more of EGF, bFGF, HGF, platelet-derived factor, triiodothyronine, and dexamethasone can enhance the related gene expression or proliferation rate of the cultured hepatocytes.

The above-obtained passaged hepatocytes were continued to culture and passage, and the ideal cell morphology could be maintained. Through identification, the hepatocytes maintained, proliferated, and passaged can be stably cultured for more than 4 months and maintaining substantially consistent morphology and function of the freshly isolated hepatocytes.

4. Passage of human primary hepatocyte in medium I-1.1/2 and I-2.1/2

Medium I-1.1 was a culture medium obtained by adding GSK3 beta inhibitor CHIR99021 and TGF beta inhibitor SB431542 into hepatocyte basal medium. Medium I-1.2 was a culture medium obtained by adding GSK3 beta inhibitor CHIR99021 and TGF beta inhibitor A83-01 into hepatocyte basal medium. The medium I-2.1/2 is a medium obtained by adding GSK3 beta inhibitor CHIR99021 and TGF beta inhibitor A83-01 and additionally NAC and OSM into hepatocyte basic medium.

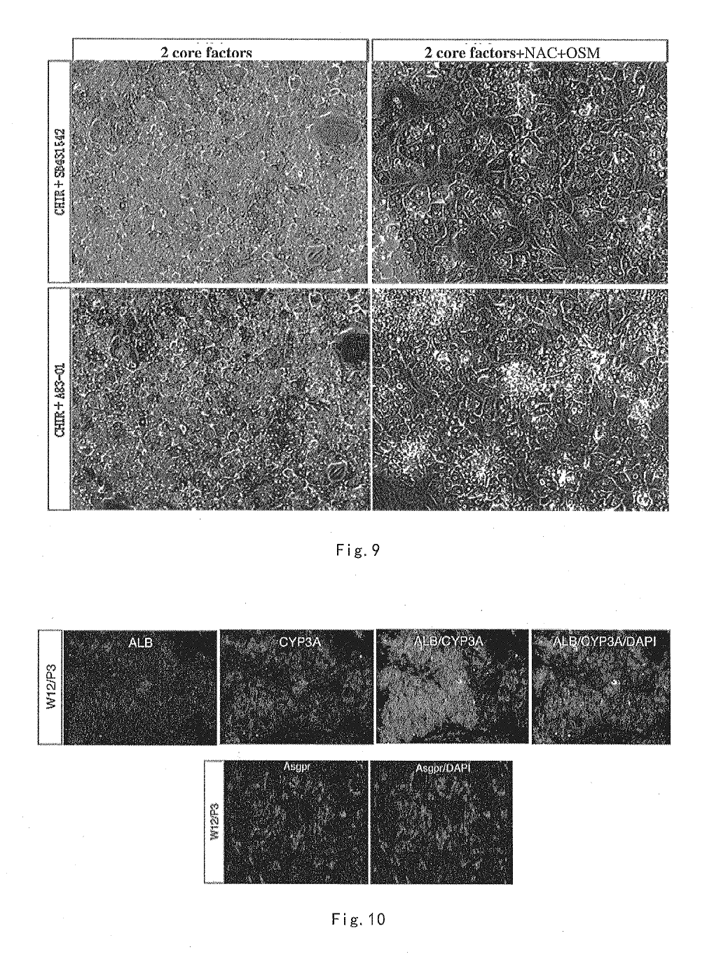

Human primary hepatocytes were cultured in culture medium I-1.1/2 and I-2.1/2 respectively. The culture method is the same as steps (2) and (3) in the aforementioned “2”. Cell morphology photo of culturing at day 20 is shown in FIG. 9.

### Example 3 Characterization of Hepatocyte

(1) Immuno-Staining

Immuno-staining method is as follows:

1. Discarding the cell culture solution, rinsing with PBS for one time,

2. Fixing with 4% Paraformaldehyde for 10 minutes, rinsing in PBS for 5 minutes×3 times,

3. Blocking with 10% goat serum for 60 min at room temperature;

4. 0.2% Triton: 25-30 min,

5. Incubating with the primary antibody (rabbit anti-ALB antibody, mouse anti-CYP3A antibody or a rabbit anti-ASGPR antibodies) for 1 hour at room temperature, or 4° C. overnight,

6. Rinsing in PBS for 5 minutes×3 times,

7. Incubating with the secondary antibody (Cy3-labeled goat anti-rabbit antibody, a FITC-labeled goat anti-mouse or goat anti-rabbit antibody) at room temperature for 45 to 60 minutes,

8. Washing with PBS for 5 min×3 times,

9. Sealing the slice, and photos were taken under fluorescence microscope.

The hepatocytes were cultured in vitro in hepatocyte culture medium I-2.2 for 84 days and the liver-specific marker was immunostained. The results are shown in FIG. 10.

From the results it can be seen that after long-term maintenance and culture, hepatocytes cultured by the present medium have significant visible hepatocyte specific markers, which means that the hepatocyte characteristics were still kept.

(2) Gene Expression Identification

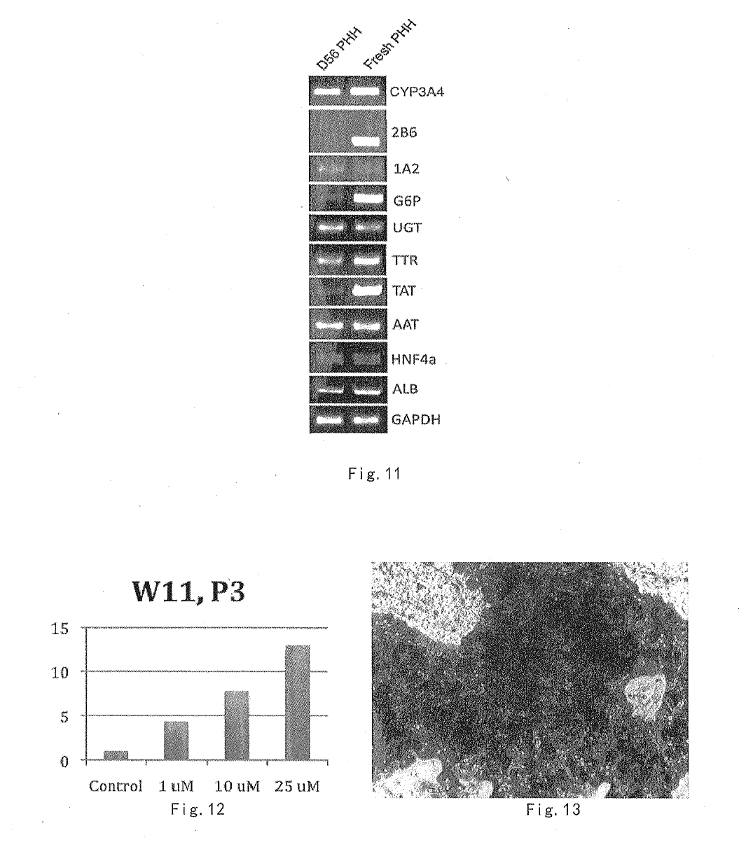

The hepatocytes were cultured in vitro in hepatocyte culture medium I-2.1 for 77 days and the liver-related gene expression was analyzed by PCR. The results are shown in FIG. 11.

From the results, it can be seen that in the hepatocytes (D56PHH) through long-term culture, the expression of genes such as ALB, HNF4a, AAT, TTR, UGT, CYP3A4, 1A2, 2B6 have almost the same level of the genes in freshly isolated hepatocytes (Fresh PHH).

(3) P450 Enzyme Induction Function Assay

Human primary hepatocytes were cultured in hepatocyte culture medium I-3.1 for 77 days (after 11 weeks). The enzyme activity of human primary hepatocytes CYP3A4 increased by induction of Rifampicine was identified.

Human primary hepatocytes were cultured in hepatocyte culture medium I-3.1 for 77 days. Cells were treated with different concentrations of rifampicin (1 μM, 10 μM, 25 μM), control groups processed the same without the addition of rifampicin were used as control. Promegap 450-Glo™ assays kit was used to detect CYP3A4 enzyme activity of the human primary hepatocytes in vitro cultured for 77 days. The elevated CYP3A4 enzyme activity under induction of rifampicin (Rifampicine) was identified.

The results are shown in FIG. 12. It can be seen that the rifampicin-induced CYP3A4 enzyme activity in the cultured human primary hepatocytes was significantly increased.

(4) Glycogen Staining:

Human primary hepatocytes were cultured in hepatocyte culture medium I-3.1 for 77 days (after 11 weeks), and stained for glycogen. Schiff assay is applied for liver glycogen staining. The detailed methods were:

1. Discarding the cell culture solution, and rinsing with PBS once;

2. Fixing with 4% Paraformaldehyde for 10 minutes, and rinsing in PBS for 5 minutes×3 times;

3. PASI was added for 10 minutes, and washed with flowing water;

4. Adding PASII solution for 1 to 2 minutes, and washing with flowing water;

5. Taking photos under microscope.

The results are shown in FIG. 13. The cultured cells were stained positive for liver glycogen, indicating that the hepatocytes retained substantially same characteristics of the freshly isolated human hepatocytes.

In the present invention all references cited are herein incorporated by reference, as if each of them is individually cited as reference. In addition, it is appreciated that, in view of the above described teaching of the invention, those skilled in the art can be made to the present invention, various changes or modifications, such equivalent forms also fall in this application is defined by the appended claims.

What is claimed is:

1. A method for culturing hepatocytes, involving maintenance, proliferation, and passaging thereof, wherein the method comprising:
culturing hepatocytes by a culture medium,
wherein said culture medium comprises:
a hepatocyte basic medium;
a GSK-3 beta inhibitor at a concentration of 0.5-10 μM, said GSK-3 beta inhibitor is CHIR99021;
a TGF beta inhibitor at a concentration of 0.5-10 μM, said TGF beta inhibitor is SB 431542 or A83-01;
N-acetyl-cysteine at a concentration of 0.25-25 mM; and
Oncostatin M at a concentration of 1-100 ng/mL,
wherein there is no exogenous gene being introduced into the hepatocytes, and the genetic background of the hepatocytes is not changed.

2. The method according to claim 1, wherein the method for culturing hepatocytes comprises:
(1) backing a culture plate for 1 to 24 hours; mixing and suspending primary hepatocytes in an adherent culture; plating; and incubating at 37° C. for 1 to 12 hours;
(2) discarding the adherent culture used in (1) above, transferring the hepatocytes into the culture medium of claim 1, and culturing the hepatocytes.

3. The method according to claim 1, wherein the hepatocyte basic medium is supplemented with 10% serum.

4. The method according to claim 1, wherein the concentration of GSK-3 beta inhibitor is 1-4 μM, and the concentration of TGF beta inhibitor is 1-8 μM.

5. The method according to claim 1, wherein the concentration of N-acetyl-cysteine is 0.5-12.5 mM.

6. The method according to claim 1, wherein the concentration of Oncostatin M is 5-80 ng/mL.

7. The method according to claim 1, wherein the culture medium further comprises an ingredient selected from the group consisting of:
human epidermal growth factor at a concentration of 5-100 ng/ml;
human fibroblast growth factor at a concentration of 5-100 ng/ml;
human hepatocyte growth factor at a concentration of 5-100 ng/ml;
dexamethasone at a concentration of 0.05-1 μM;
platelet derived factor at a concentration of 1-100 ng/ml; and
triiodothyronine at a concentration of 1-100 ng/ml.

8. The method according to claim 1, wherein the culture medium further comprises sodium pyruvate.

9. The method according to claim 1, wherein the hepatocytes are human hepatocytes.
